(19) United States
(12) Patent Application Publication
(10) Pub. No.: US 2023/0399306 A1
(43) Pub. Date: Dec. 14, 2023

(54) METHOD FOR SELECTIVELY SYNTHESIZING 3,6'-DITHIOPOMALIDOMIDE FROM POMALIDOMIDE

(71) Applicant: Aevis Bio, Inc., Daejeon (KR)

(72) Inventors: Dong Seok KIM, Daejeon (KR); Yong Jun CHOI, Chungcheongbuk-do (KR)

(21) Appl. No.: 18/033,608

(22) PCT Filed: Oct. 26, 2021
(86) PCT No.: PCT/KR2021/015129

§ 371 Date: Apr. 25, 2023

(30) Foreign Application Priority Data
Oct. 26, 2020 (KR) 10-2020-0139372

(51) Int. Cl. C07D 401/04

(52) U.S. Cl. CPC C07D 401/04

(57) ABSTRACT

In a method of synthesizing 3,6′-dithiopomalidomide from pomalidomide according to an embodiment, pomalidomide is dissolved by adding a solvent thereto, phosphorus pentasulfide (P₂S₅) is added to the dissolved pomalidomide, followed by stirring, and solids are removed from the stirred solution, followed by purification. It is possible to reduce the production of 1,6-dithiopomalidomide, which is a by-product produced during the synthesis of 3,6′-dithiopomalidomide, and selectively increase the proportion of 3,6-dithiopomalidomide among synthesized isomeric compounds to 90% or more, thereby reducing the time and cost required to separate 1,6-dithiopomalidomide by HPLC, etc. in a subsequent purification process, thereby increasing the production and economic feasibility of the 3,6′-dithiopomalidomide compound.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/015129 filed on Oct. 26, 2021, which claims priority to the benefit of Korean Patent Application No. 10-2020-0139372 filed in the Korean Intellectual Property Office on Oct. 26, 2020, the entire contents of which are incorporated herein by reference.

### BACKGROUND

### 1. Technical Field

The present invention relates to a method of selectively synthesizing 3,6′-dithiopomalidomide from pomalidomide.

### 2. Background Art

Pomalidomide (C₁₃H₁₁N₃O₄) is a derivative of thalidomide, which is a therapeutic agent for multiple myeloma, and it is used for relapsed or refractory multiple myeloma and has been studied as a therapeutic agent for various types of cancer. Pomalidomide is a type of immunomodulatory agent that modulates the immune system to kill cancer cells, and is a type of antiangiogenic inhibitor that inhibits angiogenesis necessary for cancer growth.

The present inventors previously produced various compounds based on pomalidomide and conducted studies on the pharmacological activities of these compounds, and as a result, fund that 3,6′-dithiopomalidomide may be applied for the treatment of a wide range of cancer diseases and has better effects while having less side effects than pomalidomide. Further, the present inventors have conducted studies on the excellent anticancer effect of 3,6′-dithiopomalidomide.

Meanwhile, researchers have used Lawesson's reagent (IUPAC name: 2,4-bis(4-methoxyphenyl)-1,3,2,4-dithiadiphosphetane-2,4-dithione) in the process of producing 3,6′-dithiopomalidomide from pomalidomide. The Lawesson's reagent was introduced to transformation in organic chemistry in 1968 and has been together with many reactants such as amides and ketones to perform thionation reactions.

However, Lawesson's reagent as a thionating agent has many problems. For example, Lawesson's reagent has poor thermal stability and decomposes above 110° C. In addition, Lawesson's reagent generally has low solubility, and thus it is often necessary to use hexamethylphosphoramide (HMPA) as a solvent. HMPA is suspected of being carcinogenic to humans, and the use thereof has been banned in many countries. Additional problems with Lawesson's reagents are the strong and unpleasant odor of the compound itself and the tendency to form foul-smelling by-products in the course of reaction that are difficult to separate from the desired reaction product.

In addition, when dithiopomalidomide is produced from pomalidomide using Lawesson's reagent, there are disadvantages in that the yield is low and in that 1,6-dithiopomalidomide that is an isomer is present as a major product together several by-products, and thus the process of purifying the desired product is complicated. In particular, there is a disadvantage in that up to about 90% of 1,6′-dithiopomalidomide is produced together with 3,6′-dithiopomalidomide in this process, and thus it is difficult to synthesize 3,6′-dithiopomalidomide with high purity and it takes a lot of time and money to separate 1,6′-dithiopomalidomide.

Accordingly, the synthetic method of performing the thionation reaction of pomalidomide using conventional Lawesson's reagent has a problem in that it is an inefficient process due to the production of a high percentage of 1,6′-dithiopomalidomide, and results in unacceptably low yields for commercial use and unacceptably high production costs. Therefore, it is necessary in the art to develop a new method of selectively synthesizing high-purity 3,6′-dithiopomalidomide from pomalidomide.

### SUMMARY

Therefore, a main object of the present invention is to provide a method of selectively synthesizing 3,6′-dithiopomalidomide from pomalidomide with high yield and low production cost, which may be used in commercial applications.

Another object of the present invention is to provide a method of synthesizing 3,6′-dithiopomalidomide from pomalidomide, which may increase the production yield of 3,6′-dithiopomalidomide, thereby solving the disadvantages of requiring a lot of time and cost for the separation of 1,6-dithiopomalidomide.

Other objects and advantages of the present invention will become more apparent from the following detailed description of the invention, the appended claims and the accompanying drawings.

In accordance with one aspect of the present invention, there is provided a method of synthesizing 3,6′-dithiopomalidomide from pomalidomide, the method including: a first step of dissolving pomalidomide by adding a solvent thereto; a second step of adding phosphorus pentasulfide (P₂S₅) to the dissolved pomalidomide, followed by stirring; and a third step of removing solids from the stirred solution, followed by purification.

In the method of synthesizing 3,6′-dithiopomalidomide according to the present invention, the solvent that is used in the first step may be any one solvent selected from among dioxane, toluene, and tetrahydrofuran.

In the method of synthesizing 3,6′-dithiopomalidomide according to the present invention, the dissolving in the first step may be performed by dissolving pomalidomide in the solvent at a concentration of 0.005 g/mL to 0.02 g/mL.

In the method of synthesizing 3,6′-dithiopomalidomide according to the present invention, the adding of phosphorus pentasulfide in the second step may be performed so that the molar ratio between pomalidomide and phosphorus pentasulfide is 1:1 to 1:2.5.

In the method of synthesizing 3,6′-dithiopomalidomide according to the present invention, the stirring in the second step may be performed at 105° C. to 115° C. for 20 minutes to 120 minutes.

As described above, the present invention is intended to provide a method of synthesizing 3,6′-dithiopomalidomide with high yield and low production cost, which may be used in commercial applications.

According to the method of the present invention, it is possible to reduce the production of 1,6-dithiopomalidomide, which is a by-product produced during the synthesis of 3,6′-dithiopomalidomide, and selectively increase the proportion of 3,6-dithiopomalidomide among synthesized isomeric compounds to 90% or more, thereby reducing the time and cost required to separate 1,6-dithiopomalidomide by HPLC, etc. in a subsequent purification process, thereby increasing the production and economic feasibility of the 3,6′-dithiopomalidomide compound.

### BRIEF DESCRIPTION OF THE DRAWINGS

FIG. 1 is a schematic view illustrating a chemical reaction in a process of synthesizing 3,6′-dithiopomalidomide from pomalidomide.

FIG. 2 is a schematic view illustrating a process of synthesizing pomalidomide from 3-nitrophthalic anhydride and (2,6-dioxopiperidine-3-yl)amine trifluoroacetate.

FIG. 3 is a graph showing the results of HPLC analysis of components in the product produced by the method of synthesizing 3,6′-dithiopomalidomide from pomalidomide according to the present invention.

FIG. 4 is a graph showing the results of HPLC analysis of components in the product produced by a conventional method of synthesizing 3,6′-dithiopomalidomide from pomalidomide using Lawesson's reagent.

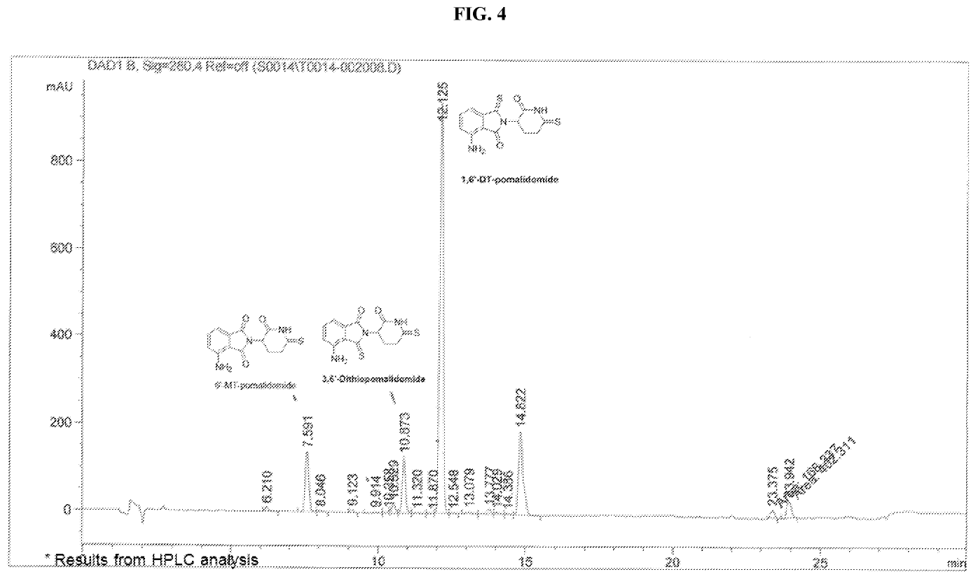

### DETAILED DESCRIPTION

In accordance with one aspect of the present invention, there is provided a method of synthesizing 3,6′-dithiopomalidomide from pomalidomide, the method including: a first step of dissolving pomalidomide by adding a solvent thereto; a second step of adding phosphorus pentasulfide (P₂S₅) to the dissolved pomalidomide, followed by stirring; and a third step of removing solids from the stirred solution, followed by purification.

3,6′-dithiopomalidomide [4-amino-2-(2-oxo-6-sulfanylidenepiperidin-3-yl)-3-sulfanylidene-2,3-dihydro-1H-isoindol-1-one] which is synthesized by the method of the present invention is a compound having the following Formula 1 and may be synthesized from pomalidomide of the following Formula 2, and 1,6′-dithiopomalidomide [7-amino-2-(2-oxo-6-sulfanylidenepiperidin-3-yl)-3-sulfanylidene-2,3-dihydro-1H-isoindol-1-one] of the following Formula 3, which is an isomer, is also synthesized in this process.

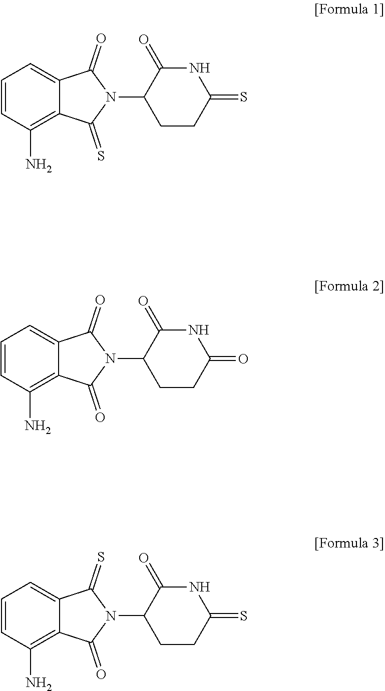 [SMILES: Nc1cccc2c1C(=S)N(C1CCC(=S)NC1=O)C2=O ; Nc1cccc2c1C(=O)N(C1CCC(=O)NC1=O)C2=O ; Nc1cccc2c1C(=O)N(C1CCC(=S)NC1=O)C2=S]

The present inventors attempted to synthesize 3,6′-dithiopomalidomide from pomalidomide using Lawesson's reagent. However, the method of synthesizing 3,6′-dithiopomalidomide using Lawesson's reagent had disadvantages in that it has a low yield and in that 1,6′-dithiopomalidomide as an isomer is synthesized as a major compound together with several by-products, and thus it is difficult to separate and purify only 3,6′-dithiopomalidomide synthesized in small amounts. This is because when 1,6′-dithiopomalidomide is synthesized as a major compound and a separation device such as PREP-HPLC is used, 3,6′-dithiopomalidomide containing impurities in the region where 1,6′-dithiopomalidomide and 3,6′-dithiopomalidomide are eluted simultaneously cannot be used, and thus the yield is further reduced.

In addition, a major problem encountered in the thionation reaction of pomalidomide using Lawesson's reagent is that Lawesson's reagent generally reacts at the most accessible position. That is, the carbonyl group at the 6′ position among the two thionation reaction positions that are synthesized into 3,6′-dithiopomalidomide easily undergoes a thionation reaction. Unlike this, the carbonyl group at the 3′ position, which is the other one close to the aniline amine located in the phthalimide-imide carbonyl group, is sterically encumbered, preventing access of a thionating agent. Thus, the thionation reaction for the carbonyl group at the 1′ position proceeds preferentially, and for this reason, when the thionation reaction is performed using Lawesson's reagent, the synthesis of 1,6′-dithiopomalidomide significantly increases (at least 90%) compared to that of 3,6′-dithiopomalidomide.

In order to overcome this problem, the present inventors have studied various thionation reactions that preferentially thionate the carbonyl group at the 3′ position of pomalidomide, and as a result, have found that, when phosphorous pentasulfide (P₂S₅) is applied as a thionating agent for pomalidomide, the steric encumbrance of the carbonyl group at the 3′ position can be overcome and the thionation reaction can proceed preferentially at the 3′ position, whereby, among two isomers (3,6′-dithiopomalidomide and 1,6′-dithiopomalidomide), 3,6′-dithiopomalidomide can be selectively produced.

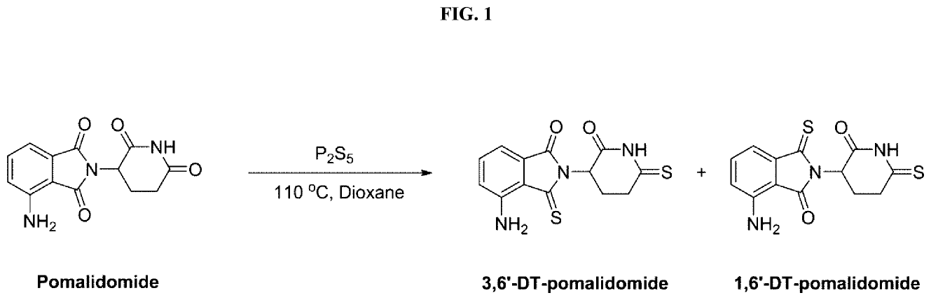

Referring to FIG. 1, in the method of synthesizing 3,6′-dithiopomalidomide from pomalidomide according to the present invention, phosphorus pentasulfide (P₂S₅) is added dropwise to pomalidomide dissolved in a solvent and stirred to synthesize 3,6′-dithiopomalidomide, and the proportion of 3,6′-dithiopomalidomide among the two isomers (3,6′-dithiopomalidomide and 1,6′-dithiopomalidomide) produced in this process is 90% or more.

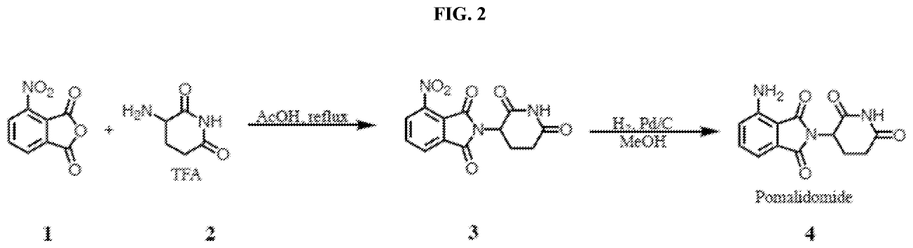

Meanwhile, as the pomalidomide used in the present invention, a commercially available compound may be used, but pomalidomide produced by direct synthesis may also be used. For direct synthesis of pomalidomide that may be used in the present invention, 3-nitrophthalic anhydride (1.5 g, 7.8 mmol) and (2,6-dioxopiperidine-3-yl)amine trifluoroacetate (1.9 g, 7.8 mmol) are reacted with stirring in acetic acid (60.0 mL) as a solvent for 4.5 hours under a nitrogen atmosphere to produce a 2-(2,6-dioxopiperidine-3-yl)-4-nitrophthalimide compound which was then reacted with 2-(2, 6-dioxopiperidine-3-yl)-4-nitrophthalimide (66.0 mg, 0.2 mmol) in methanol (80.0 mL) as a solvent in the presence of palladium (10 wt %, 45.0 mg) on activated carbon at room temperature under a hydrogen atmosphere (44 lbs) for 7 hours (see FIG. 2).

In the first step, phosphorus pentasulfide as a thionating agent and pomalidomide as a compound to be thionated may be reacted in a liquid solvent medium for the thionating agent. That is, pomalidomide, a compound that is to be thionated with the thionating agent phosphorus pentasulfide, may be dissolved in a liquid solvent medium, and the solvent may be any one solvent selected from among dioxane, toluene, and tetrahydrofuran. In a preferred example of the present invention, the thionation reaction was performed using dioxane as a solvent. After the reaction is performed using a solvent such as dioxane, toluene, or tetrahydrofuran, the remaining salt produced by the decomposition of phosphorus pentasulfide may be easily separated from the thionated compound using a filter after lowering the reaction temperature to room temperature. Further purification of the reaction product may optionally be performed, for example, by recrystallization.

In the method of synthesizing 3,6′-dithiopomalidomide according to the present invention, the dissolution in the first step may be performed by dissolving pomalidomide in a solvent at a concentration of 0.005 g/mL to 0.02 g/mL. Phosphorus pentasulfide as a thionating agent and pomalidomide as a compound to be thionated are reacted in a liquid solvent medium for the thionating agent, and the concentration of pomalidomide is preferably adjusted so that pomalidomide may be sufficiently dissolved in the solvent medium. In a preferred example of the present invention, 1 g (3.66 mmol) of pomalidomide was after sufficiently dissolved in 100 mL of dioxane as a solvent, and then the thionating agent phosphorus pentasulfide was added to and reacted with the pomalidomide solution. It could be confirmed that, when the solvent was used at the above-described concentration, the rate of conversion to 3,6′-dithiopomalidomide was 90% or more.

In addition, in the method of synthesizing 3,6′-dithiopomalidomide according to the present invention, the addition of phosphorus pentasulfide in the second step may be performed so that the molar ratio between pomalidomide and phosphorus pentasulfide is 1:1 to 1:2.5. The addition ratio of phosphorus pentasulfide as a thionating agent to pomalidomide as a compound to be thionated affects the yield of the reaction product. Since the number of carbonyl groups in pomalidomide that are thionated to form 3,6′-dithiopomalidomide is two, it is ideal that pomalidomide and phosphorus pentasulfide are added at a ratio of 1:2 and reacted with each other. However, in consideration of the fact that various by-products are produced during the reaction process, it is preferable to add the thionating agent in excess of the molar amount of pomalidomide which is a compound to be thionated. Thus, it is preferable to add pomalidomide and phosphorus pentasulfide at a molar ratio of 1:1 to 1:2.5. If pomalidomide and phosphorus pentasulfide are added at a molar ratio of less than 1:1, the thionating agent, the thionation reaction will not be sufficiently performed because the amount of thionating agent phosphorus pentasulfide added is not sufficient, and if pomalidomide and phosphorus pentasulfide are added at a ratio of more than 1:2.5, the phosphorus pentasulfide added will cause an excessive thionation reaction, resulting in an increase in the production of by-products, which results in a decrease in the yield of the 3,6′-dithiopomalidomide compound.

In addition, in the method of synthesizing 3,6′-dithiopomalidomide according to the present invention, the second step of adding phosphorus pentasulfide to pomalidomide, followed by stirring, may be performed at 105° C. to 115° C. for 20 minutes to 120 minutes. If the reaction temperature in the stirring step is lower than 105° C., there is a disadvantage in that the yield of the 3,6′-dithiopomalidomide compound is lowered because sufficient heat energy required for the reaction to occur is not supplied, and if the reaction temperature is higher than 115° C., the amount of by-products produced will increase, resulting in a decrease in the yield of the 3,6′-dithiopomalidomide compound. In addition, if the reaction time is shorter than 20 minutes, there is a disadvantage in that the yield of the 3,6′-dithiopomalidomide compound is lowered because a sufficient thionation reaction does not occur, and if the reaction time is longer than 120 minutes, there is a disadvantage in that economic efficiency is not achieved because a sufficient reaction has already proceeded.

Hereinafter, the present invention will be described in more detail with reference to examples. These examples are intended merely to illustrate the present invention, and the scope of the present invention is not to be construed as being limited by these examples.

[Experimental Materials and Analytical Methods]

The materials used in the experiments are shown in Table 1 below.

TABLE 1

| Material | Supplier/Cat. No. | Lot No. |
|---|---|---|
| Pomalidomide | Combi Block, QB-4271 | B41848 |
| Phosphorus | Aldrich, 232106 | MKCK6600 |
| pentasulfide (P₂S₅) | | |
| Dioxane | SAMCHUN, D0645 | 82416 |
| Chloroform (CHCl₃) | SAMCHUN, C0585 | 72420 |
| Acetone | SAMCHUN, A0098 | 80520 |
| DMSO-d₆ | ARMAR, 300497 | 1004323 |
| Silica gel (SiO₂) | MERCK, 1.09385.2500 | TA0542185849 |

NMR spectra were recorded on a Bruker BioSpin 400 MHz (AVANCE III 400) at room temperature. Analytical samples were dissolved in DMSO-d₆ and analyzed using NMR tubes (5-mm o.d., Wilmad, WG-1000-7). In addition, mass spectrometry was performed using a Waters SQ Detector 2 instrument operated in the ESI (+) ionization mode.

HPLC analysis was performed using an Agilent 1100 series instrument.

### Example 1. Reaction of Pomalidomide:Phosphorus Pentasulfide=1:2 (mol); 20 Minutes

Pomalidomide (1 g, 3.66 mmol) was dissolved in dioxane (100 mL, 100 v/g), and then phosphorus pentasulfide (P₂S₅) (1.63 g, 7.32 mmol) was added dropwise thereto, followed by stirring at 110° C. for 20 minutes. Immediately after lowering the reaction temperature to room temperature, solids were removed using a filter, and the solvent was concentrated under reduced pressure to obtain a reaction product.

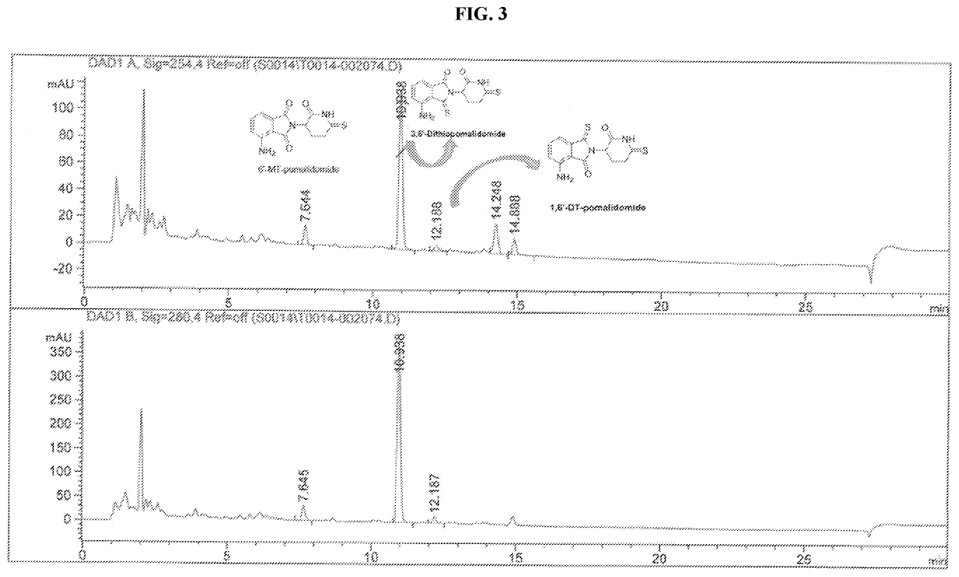

As a result of HPLC and NMR analysis using 0.5 mg/mL of the reaction product as a sample, it was determined that the yield of the sum of compounds (3,6′-dithiopomalidomide and 1,6′-dithiopomalidomide) was 41%, and the ratio between these compounds was 98:2 (see FIG. 3).

¹H-NMR (DMSO-d6) d12.61 (s, 1H, NH), 7.63 (s, br, 2H, NH₂), 7.47 (t, J=7.6 Hz, 1H, C₆—H), 7.16 (d, J=8.6 Hz, 1H, C₇—H), 7.01 (s, br, 1H, C₅—H), 5.76 (s, br, 1H, C3′-H), 3.20-2.80 (m, 3H) and 2.05-1.99 (m, 1H) ppm; ¹³C NMR (DMSO-d6) d 210.7, 194.4, 170.0, 167.5, 148.5, 135.3, 128.1, 124.2, 114.6, 112.2, 50.5, 41.5 and 24.0 ppm; GCMS (CI/CH₄), m/z 305 (M+)

In addition, the entire reaction product obtained by performing the same reaction under the above reaction conditions was purified by column chromatography using silica gel (CHCl₃:acetone=100:5). In this process, the 3,6′-dithiopomalidomide detection region overlapping the 1,6′-dithiopomalidomide detection region was excluded, and only the pure 3,6′-dithiopomalidomide detection region was recovered to obtain 100% pure 3,6′-dithiopomalidomide. The separated 3,6′-dithiopomalidomide was recrystallized from DCM (20 v/g) to obtain 3,6′-dithiopomalidomide (325 mg, T0027-05) as a dark orange solid in a yield of 29.1%.

### Example 2. Reaction of Pomalidomide:Phosphorus Pentasulfide=1:2 (mol); 60 Minutes

Pomalidomide (1 g, 3.66 mmol) was dissolved in dioxane (100 mL, 100 v/g), and then phosphorus pentasulfide (P₂S₅) (1.63 g, 7.32 mmol) was added dropwise thereto, followed by stirring at 110° C. for 60 minutes. Immediately after lowering the reaction temperature to room temperature, solids were removed using a filter, and the solvent was concentrated under reduced pressure to obtain a reaction product.

As a result of HPLC analysis using 0.5 mg/mL of the reaction product as a sample, it was determined that the yield of the sum of compounds (3,6′-dithiopomalidomide and 1,6′-dithiopomalidomide) was 19%, and the ratio between these compounds was 99:1.

### Example 3. Reaction of Pomalidomide:Phosphorus Pentasulfide=1:2 (mol); 960 Minutes

Pomalidomide (1 g, 3.66 mmol) was dissolved in dioxane (100 mL, 100 v/g), and then phosphorus pentasulfide (P₂S₅) (1.63 g, 7.32 mmol) was added dropwise thereto, followed by stirring at 110° C. for 960 minutes. Immediately after lowering the reaction temperature to room temperature, solids were removed using a filter, and the solvent was concentrated under reduced pressure to obtain a reaction product.

As a result of HPLC analysis using 0.5 mg/mL of the reaction product as a sample, it was determined that the yield of the sum of compounds (3,6′-dithiopomalidomide and 1,6′-dithiopomalidomide) was 10%, and the ratio between these compounds was 99:1.

### Example 4. Reaction of Pomalidomide:Phosphorus Pentasulfide=1:1 (mol); 30 Minutes

Pomalidomide (1 g, 3.66 mmol) was dissolved in dioxane (100 mL, 100 v/g), and then phosphorus pentasulfide (P₂S₅) (0.815 g, 3.66 mmol) was added dropwise thereto, followed by stirring at 110° C. for 30 minutes. Immediately after lowering the reaction temperature to room temperature, solids were removed using a filter, and the solvent was concentrated under reduced pressure to obtain a reaction product.

As a result of HPLC analysis using 0.5 mg/mL of the reaction product as a sample, it was determined that the yield of the sum of compounds (3,6′-dithiopomalidomide and 1,6′-dithiopomalidomide) was 18%, and the ratio between these compounds was 91:9.

### Example 5. Reaction of Pomalidomide:Phosphorus Pentasulfide=1:1 (mol); 60 Minutes

Pomalidomide (1 g, 3.66 mmol) was dissolved in dioxane (100 mL, 100 v/g), and then phosphorus pentasulfide (P₂S₅) (0.815 g, 3.66 mmol) was added dropwise thereto, followed by stirring at 110° C. for 60 minutes. Immediately after lowering the reaction temperature to room temperature, solids were removed using a filter, and the solvent was concentrated under reduced pressure to obtain a reaction product.

As a result of HPLC analysis using 0.5 mg/mL of the reaction product as a sample, it was determined that the yield of the sum of compounds (3,6′-dithiopomalidomide and 1,6′-dithiopomalidomide) was 28%, and the ratio between these compounds was 93:7.

### Example 6. Reaction of Pomalidomide:Phosphorus Pentasulfide=1:1 (mol); 120 Minutes

Pomalidomide (1 g, 3.66 mmol) was dissolved in dioxane (100 mL, 100 v/g), and then phosphorus pentasulfide (P₂S₅) (0.815 g, 3.66 mmol) was added dropwise thereto, followed by stirring at 110° C. for 120 minutes. Immediately after lowering the reaction temperature to room temperature, solids were removed using a filter, and the solvent was concentrated under reduced pressure to obtain a reaction product.

As a result of HPLC analysis using 0.5 mg/mL of the reaction product as a sample, it was determined that the yield of the sum of compounds (3,6′-dithiopomalidomide and 1,6′-dithiopomalidomide) was 38%, and the ratio between these compounds was 94:7.

### Comparative Example. Synthesis of 3,6′-dithiopomalidomide Using Lawesson's Reagent

3,6′-dithiopomalidomide was synthesized according to a conventional synthesis method using Lawesson's reagent.

Pomalidomide (100 mg, 0.366 mmol) and Lawesson's reagent (81.3 mg, 0.201 mmol) dissolved in anhydrous toluene were refluxed under a nitrogen atmosphere for 6 hours. Then, Lawson's reagent (162.6 mg, 0.402 mmol) was further added, and the reaction mixture was refluxed for 16 hours.

As a result of HPLC analysis using 0.5 mg/mL of the reaction product as a sample, it was determined that the yield of the sum of compounds (3,6′-dithiopomalidomide and 1,6′-dithiopomalidomide) was 16.1%, and the ratio between these compounds was 10:90.

The experimental results of the Examples and the Comparative Example are summarized in Table 2 below.

TABLE 2

| | Reaction time | | Yield | 3,6′-DTP:1,6′-DTP |
|---|---|---|---|---|
| Example 1 | 20 | min | 41% | 98:2 |
| Example 2 | 60 | min | 19% | 99:1 |
| Example 3 | 960 | min | 10% | 99:1 |
| Example 4 | 30 | min | 18% | 91:9 |
| Example 5 | 60 | min | 28% | 93:7 |
| Example 6 | 120 | min | 38% | 94:6 |

| | | | |
|---|---|---|---|
| Comparative | — | 16% | 10:90 |
| Example | | | |

Referring to Table 2 above, it could be confirmed that, when pomalidomide and phosphorus pentasulfide were mixed and reacted without using Lawesson's reagent, the proportion of 3,6′-dithiopomalidomide relative to the sum of 3,6′-dithiopomalidomide and 1,6′-dithiopomalidomide produced was predominant (90% or more), and thus the amount of 3,6′-dithiopomalidomide finally obtained in the subsequent purification process could be dramatically increased.

With further reference to the foregoing detailed description and illustrative examples, a person skilled in the art can understand that the present invention can be implemented without departing from the scope of the appended claims using ordinary experimentation in order to perform a thionation reaction, for example, on the carbonyl group at the 3,6′-position among the four carbonyl groups present in pomalidomide to be thionated. For example, the reaction may be performed at normal ambient pressure or under an inert atmosphere such as argon or nitrogen. Other parameters that can be optimized or varied are, for example, solvent medium, reaction temperature, and reaction time, and all such modifications and variations are contemplated as being within the scope of the present invention.

1: A method of synthesizing 3,6′-dithiopomalidomide from pomalidomide, the method comprising:
dissolving pomalidomide by adding a solvent thereto;
adding phosphorus pentasulfide (P₂S₅) to the dissolved pomalidomide, followed by stirring; and
removing solids from the stirred solution, followed by purification.

2: The method of claim 1, wherein the solvent in the dissolving is any one solvent selected from the group consisting of among dioxane, toluene, and tetrahydrofuran.

3: The method of claim 1, wherein the dissolving is performed by dissolving pomalidomide in the solvent at a concentration of 0.005 g/mL to 0.02 g/mL.

4: The method of claim 1, wherein, in the adding, a molar ratio between pomalidomide and phosphorus pentasulfide is 1:1 to 1:2.5.

5: The method of claim 1, wherein the stirring is performed at 105° C. to 115° C. for 20 minutes to 120 minutes.
